(19) United States
(12) Patent Application Publication
(10) Pub. No.: US 2024/0252426 A1
(43) Pub. Date: Aug. 1, 2024

(54) IMPROVED AND STABLE APREMILAST PHARMACEUTICAL COMPOSITIONS

(71) Applicant: APRAMITHA INNOVATIONS PRIVATE LIMITED, Secunderabad, Telangana (IN)

(72) Inventors: Venkata Nookaraju SREEDHARALA, Hyderabad (IN); Srikanth KALAKOTI, Hyderabad (IN)

(73) Assignee: APRAMITHA INNOVATIONS PRIVATE LIMITED, Secunderabad, Telangana (IN)

(21) Appl. No.: 17/278,194

(22) PCT Filed: Sep. 20, 2019
(86) PCT No.: PCT/IN2019/050689

§ 371 Date: Mar. 19, 2021

(30) Foreign Application Priority Data
Sep. 21, 2018 (IN) 201843035551

(51) Int. Cl. A61K 9/00; A61K 31/4035; A61K 47/32

(52) U.S. Cl. CPC A61K 9/0014; A61K 31/4035; A61K 47/32

(57) ABSTRACT

The present invention relates to an improved and stable topical pharmaceutical compositions of apremilast used for the treatment of psoriasis and/or psoriatic arthritis. It further relates to processes of preparation of the stable compositions and the method of use for these compositions. The present invention provides a topical composition of Apremilast with improved and enhanced diffusion rate over prior art Apremilast topical compositions and also provides a drug diffusion form the topical composition at a lower strength which is equal to or better than a higher strength conventional composition of Apremilast.

### CROSS REFERENCE

This application claims priority from Indian Patent Application No. 201843035551 filed on Sep. 21, 2018, which is a patent of addition claiming priority from Indian patent application number 201641011015 filed on Mar. 30, 2016 which is now granted as Indian Patent No. 301416.

### FIELD OF INVENTION

The present invention provides topical pharmaceutical composition of drugs used for the treatment of psoriasis and/or psoriatic arthritis.

### BACKGROUND OF INVENTION

This invention relates to topical pharmaceutical composition of Apremilast, a method of use thereof and a method of manufacture.

Apremilast is a phosphodiesterase4 (PDE4) inhibitor. Apremilast is known chemically as N-[2-[(1S)-1-(3-ethoxy-4-methoxyphenyl)-2-(methylsulfonyl)ethyl]-2,3-dihydro-1,3-dioxo-1H-isoindol-4-yl]acetamide, having chemical structure as given below:

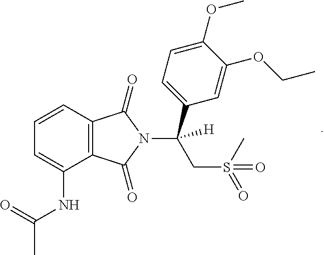 [SMILES: CCOc1cc([C@@H](CS(C)(=O)=O)N2C(=O)c3cccc(NC(C)=O)c3C2=O)ccc1OC]

Its empirical formula is C₂₂H₂₄N₂O₇S and the molecular weight is 460.5. It is an inhibitor of phosphodiesterase 4 (PDE4), is indicated for the treatment of psoriatic arthritis.

Psoriasis is a chronic, non-contagious skin disorder that appears in many different forms and can affect any part of the body. The most common type of psoriasis is plaque psoriasis, occurring in 80% of people suffering from the disease. Plaque psoriasis is characterized by red patches and lesions that are covered by a build-up of skin cells that are often called scales, and most commonly seen on the elbows, knees, scalp and back. Psoriasis is classified as mild, moderate, or severe, depending on the percentage of body surface involved and severity of the disease.

According to the National Institutes of Health, psoriasis is one of the most common human skin disorders, affecting greater than 3% of the United States population, or more than 5 million adults, of which greater than 1.5 million are considered to have a moderate to severe form of the disease. Although psoriasis is not fatal, it negatively impacts quality of life to a degree similar to heart disease and arthritis (Rapp et al. 1999). In addition, 10-30% of patients with psoriasis also develop a form of arthritis—psoriatic arthritis, which damages the bone and connective tissue around the joints. Furthermore, inflammatory mediators associated with psoriasis may increase the risk of obesity, diabetes, thrombosis, and atherosclerosis (Davidovici et al. 2010).

Apremilast is at present available only as an oral formulation, marketed by Celgene Corp, under the trade name of OTEZLAR®. OTEZLAR® tablets are supplied in 10, 20, and 30 mg strengths for oral administration. OTEZLAR® tablets are indicated (1) for the treatment of patients with active psoriatic arthritis and (2) for the treatment of patients with moderate to severe psoriasis who are candidates for phototherapy or systemic therapy.

U.S. Pat. No. 9,468,605 discloses oral formulation of Apremilast, more specifically immediate release tablets of Apremilast.

U.S. Pat. No. 7,208,516 discloses various methods for treating psoriasis and rheumatoid arthritis using oral therapies.

PCT publication no. WO2015/120110 A2 discloses extended release compositions for oral administration. PCT publication no. WO2012/083153 A1 discloses oligomer-containing Apremilast compounds and their oral compositions.

However oral therapy like have certain side effects liked diarrhea, headache, nausea, upper respiratory tract infection, vomiting, abdominal pain, indigestion/heartburn, decreased appetite, insomnia, back pain, frequent bowel movements, depression, bronchitis, tooth abscess, and sinus headache. Other side effects of oral therapy include hypersensitivity, weight loss, gastro-esophageal reflux disease (GERD), migraine, cough, and rash. Thus systemic absorption of oral dosage forms creates certain disadvantages which require further concern.

Though, Apremilast is available as an effective oral therapy for psoriasis; an oral tablet composition is less suitable due to its higher systemic absorption and for patients who have difficulty in swallowing or whose gastro intestinal side effects are not reduced even after suggested oral dose titration. Thus, there is need for improved and stable pharmaceutical composition of Apremilast suitable for topical administration that overcomes the problems associated with oral compositions.

In general Apremilast is difficult to formulate as topical composition due to its solubility issues and complexity associated with stabilization of the composition. Inventors of the present application have been involved in the research and development of pharmaceutical formulations, and through extensive research they have tried to develop a stable and improved topical composition of Apremilast.

### SUMMARY OF THE INVENTION

An object of the present invention is to provide improved and stable topical compositions of Apremilast for treatment of psoriasis or psoriatic arthritis.

A further object of the present invention is to provide a topical pharmaceutical composition in the form of topical gel or cream or ointment or solution or spray along with dimethyl sulfoxide to provide desired topical pharmacological actions and fewer side effects.

In yet another object the present invention provides a topical composition of Apremilast that can be prepared with or without carbopol, with or without Dimethyl sulfoxide (DMSO).

Another object of the present invention is to provide a topical composition of Apremilast comprising Apremilast particle that are micronized or un-micronized.

Yet another object of the present invention is to provide a topical composition of Apremilast with improved and enhanced diffusion rate over prior art Apremilast topical compositions.

In another object the invention provides a topical composition which comprises proportions of carbopol and dimethyl sulfoxide with micronized or un-micronized drug particles, for enhanced diffusion of the drug.

In yet another object of the invention, the proposed composition provides a drug diffusion form the topical composition at a lower strength which is equal to or better than a higher strength composition of prior art.

### BRIEF DESCRIPTION OF DRAWINGS

FIG. 1: particle size data of Apremilast compositions.

FIG. 2: showing effect of various parameters on drug release—

FIG. 2(a): Effect of carbopol concentration on drug release from the composition.

FIG. 2(b): Effect of permeation enhancer (DMSO) concentration on drug release from the composition.

FIG. 2(c): Effect of micronization of drug particles on drug release from the composition.

FIG. 3: PLM (Polarised light micrograph) photographs (10× magnification): 3(a) 2% Apremilast topical gel; 3(b) 4% Apremilast topical gel.

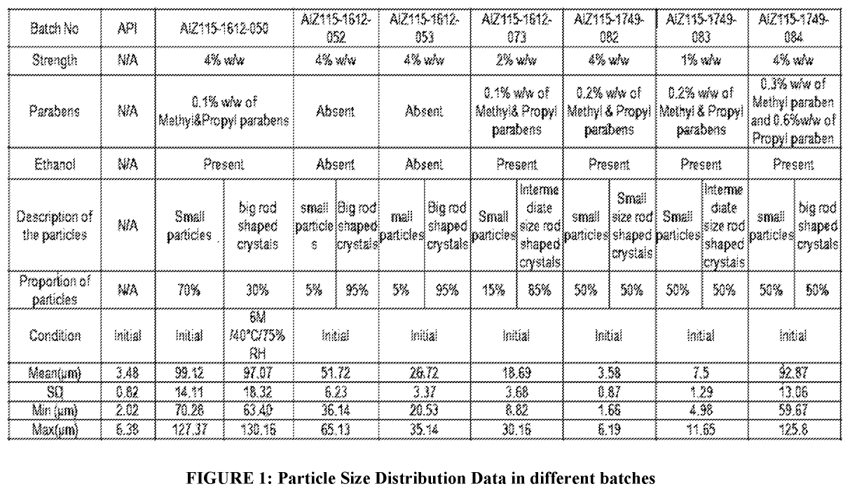

### DETAILED DESCRIPTION OF THE INVENTION

The present invention relates to improved and stable topical compositions of Apremilast for treatment of psoriasis or psoriatic arthritis.

Accordingly the present invention provides a topical pharmaceutical composition suitable for topical application, comprising Apremilast or its pharmaceutically acceptable salt in combination with pharmaceutically acceptable excipients.

It has been found that pharmaceutical compositions in the form of topical formulation of Apremilast as prepared provide desired pharmacological actions with better penetration of drug and fewer side effects.

In one embodiment of the invention, Apremilast is provided in the pharmaceutical compositions in the form of topical gel, topical cream, topical ointment, topical solution or topical spray.

In another embodiment of the invention, Apremilast is provided in the pharmaceutical composition in the form of topical gel or cream or ointment or solution or spray along with carbopol and dimethyl sulfoxide.

In another embodiment the present topical composition can be prepared with or without carbopol.

In another embodiment the present topical composition can be prepared with or without Dimethyl sulfoxide (DMSO).

In another embodiment the present topical composition can be prepared with Apremilast particle that are micronized or un-micronized.

In yet another preferred embodiment the topical composition of the present invention comprises of a pharmaceutically acceptable permeation enhancer and a carrier, wherein the composition provides better and improved effect over the other topical compositions known in the art or the compositions devoid of specific quantity or ratio of permeation enhancer or carrier as present in instant topical composition.

In a further important embodiment of the present invention, the presently proposed composition is capable of providing an enhanced diffusion of the drug from a lower strength topical composition of Apremilast over prior art higher strength topical composition of Apremilast.

In another preferred embodiment the inventors of the present invention have developed the topical pharmaceutical composition of Apremilast with enhanced diffusion rate over previously known topical compositions of Apremilast.

In yet another preferred embodiment of the invention the composition comprises of carbopol and dimethyl sulfoxide with micronized or un-micronized drug particles. The inventors have surprisingly found in the experiments that such combination in particular composition as contemplated imparts enhanced diffusion of the drug into the skin layers.

In another embodiment of the invention, the topical gel or cream or ointment or solution or spray comprises excipients that are hydrophilic in nature.

In another embodiment of the invention, the topical gel or cream or ointment or solution or spray comprises excipients that are hydrophobic in nature.

In an embodiment of the invention, topical gel or cream or ointment or solution or spray comprises excipients that are hydrophilic and/or hydrophobic in nature. In yet another embodiment of the invention, topical pharmaceutical composition comprises excipients to provide better feel to the skin, lower irritation and low or non-staining to the skin or the clothes.

In one of the embodiment of the invention, topical pharmaceutical composition may optionally comprise of fragrance.

In one embodiment, the invention is directed to a topical composition for treating psoriasis comprising a therapeutically effective amount of Apremilast and a pharmaceutically acceptable carrier.

A “therapeutically effective amount” is an amount necessary to palliate at least one symptom of psoriasis. For example, a therapeutically effective amount is sufficient to treat (i.e. alleviate or reduce) at least one of: itching/scratching, redness, inflammation, cracking, scaling, bleeding, etc. Preferably, the therapeutically effective amount of Apremilast comprises between 0.5 to 15% by weight of the composition, more preferably 1 to 10%.

The term “pharmaceutically acceptable” means approved by a regulatory agency of the Federal or state government or listed in the U.S. Pharmacopeia or other generally recognized pharmacopeia for use in animals, and more particularly in humans.

The term “carrier” refers to a diluent, adjuvant, excipient, penetration enhancer, or vehicle with which an active ingredient is administered. Such pharmaceutical carriers can be liquids, such as water and oils, including those of petroleum, animal, vegetable or synthetic origin. Preferably, the pharmaceutically acceptable carrier comprises excipients commonly used in topically applied formulations (water, oil-based lotions, sprays, ointments, gels etc.).

In an embodiment of the invention, topical composition of present invention has excipients that help deep penetration of Apremilast in to the target area of the skin and provides ease of application, spreadability and cleaning.

Topical pharmaceutical compositions of the invention include but not limited to cream, ointment, gel, solution or spray.

Topical pharmaceutical compositions of the invention include Apremilast in the concentration of 1% to 25% by weight.

Topical pharmaceutical compositions of the invention include Apremilast and DMSO in the ratio of 1:100 to 100:1 or 1:10 to 10:1 or 1:1.

Topical pharmaceutical compositions of the invention include Apremilast and carbopol in the ratio of 0.5:0.2 to 15:3, preferably 0.5:0.5 to 10:1.5.

Topical pharmaceutical compositions of the invention have a pH in the physiological range between 1 to 8 or 2 to 7 or 3 to 6.

In yet another preferred embodiment of the invention the topical composition comprises of carbopol 0.2-3%, preferably 0.3-1.5%, more preferably 0.5-1.2%; dimethyl sulfoxide, and Apremilast 0.5-15%, preferably 0.5-10%, more preferably 0.5-5%.

In yet another preferred embodiment of the invention the topical composition comprises of carbopol 0.2-3%, preferably 0.3-1.5%, more preferably 0.5-1.2%; dimethyl sulfoxide 0%, and Apremilast 0.5-15%, preferably 0.5-10%, more preferably 0.5-5%.

A Non-Limiting List of the Excipients that can be Used in the Composition are:

A pharmaceutically acceptable carrier may comprise water, glycerin, petrolatum, stearic acid, glycol stearate, dimethicone, isopropyl isostearate, synthetic and semi-synthetic starches, cetyl alcohol, glyceryl stearate, magnesium aluminum silicate, carbomer, ethylene brassylate, triethanolamine, disodium EDTA, phenoxyethanol, methyl paraben, propyl paraben, ethanol, bio-polymers (e.g., sodium hyaloronate), liposomes, nano- and micro-particulate carriers, and/or titanium dioxide. More preferably, the pharmaceutically acceptable carrier comprises dimethyl sulfoxide (DMSO), glycerol, propylene glycol, petrolatum water, and one or more pharmaceutically acceptable penetration enhancer (absorption promoter and/or accelerants).

Typically, the topical compositions of the invention comprise skin penetration enhancers, pharmaceutical surfactants and solubility enhancers, oil phase components, aqueous phase components, emulsifiers, moisturizers, antioxidants, vitamins, lubricants, preservatives, stabilizers and other ingredients.

Skin penetration enhancers reversibly decrease the barrier resistance of the skin, which increases the amount of Apremilast absorbed. Preferably, skin penetration enhancers include, but are not limited to, sulfoxides (e.g. DMSO), azones (e.g. laurocapram), pyrrolidones (e.g., 2-pyrrolidone), alcohols and alkanols (e.g., ethanol, decanol, etc.), oleic acid (and derivatives thereof), glycols (e.g., propylene glycol), dimethylformamide (DMF), dimethylacetamide (DMAC), fatty alcohols (e.g., lauryl alcohol), fatty acid esters, fatty acids, fatty alcohol ethers (e.g., EO-2-oleyl ether), terpenes, and proteins or yellow fat.

Pharmaceutical surfactants or solubility enhancers include, but are not limited to, lauryl alcohol, polyoxyethylene ether, polyoxyethylene glycerol monostearate, stearic acid ester oxygen poly hydrocarbon, vitamin E succinate polyethylene glycol ester, sorbitan esters, polyoxyethylene castor oil, polyoxyethylene hydrogenated castor oil, poloxamer, organic esters (e.g. ethylene acetate), and poly hill dinitrate 80 (i.e. Tween 80 or its mixture). In a preferred embodiment, the pharmaceutical surfactants or solubility enhancers include DMSO, polyvinylpyrrolidones, stearic acid hydrocarbon oxygen Poly (40) ester, lauryl alcohol polyoxyethylene (23) ether, vitamin E succinate polyethylene glycol ester, ethylene acetate, and polyoxyethylene (40) hydrogenated castor oil (and its mixtures, i.e. polyoxy (40) stearate). Still, in a more preferred embodiment the pharmaceutical surfactants or solubility enhancers include sodium lauryl sulphate and sorbitan esters.

Suitable oily phase may include, but are not limited to, glyceryl monoacetate, glycerol diacetate, glyceryl triacetate, stearic acid, oils, palmitic acid, and any other suitable pharmaceutically acceptable oils, oleic acid, medium-chain triglycerides, single-decane triglyceride, fat (e.g., lanolin), mineral oils, paraffin, waxes from all sources, petrolatum, hydrocarbons, vaseline, and mixtures thereof.

Aqueous phase components include, but are not limited to, de-ionized water, glycerol gelatin, cellulose derivatives (e.g., microcrystalline cellulose (Avicel PH 101)), and polyethylene glycol (PEG 300 to PEG 6000), and mixtures thereof.

Emulsifiers or thickeners include, but are not limited to oleyl alcohol, polyoxyethylene oleyl ether, PEG-40 stearate, ceteareth-12, ceteareth-20, ceteareth-30, glyceryl stearate, PEG-100 stearate, methyl myristate, isopropyl myristate, synthetic and semi-synthetic commercially available gums, Arlacel 165, glyceryl stearate, PEG-100 stearate, steareth-2 and steareth-20, dimethicone copolyol, Polysorbate 20 (Tween 20), cetyl esters wax, Polysorbate 40 (Tween 40), Polysorbate 60 (Tween 60), Polysorbate 80 (Tween 80), lauramide DEA (diethanolamine lauric acid amide), cocamide DEA (cocamide diethanolamine), and cocamide MEA (cocamide monoethanolamine), Phospholipid PTC (cocamidopropyl pg-dimonium chloride phosphate), alginate, carrageenan, Glucate DO (methyl glucose dioleate), methylcellulose, polyvinyl alcohol, Sepineo P 600, Carbopol and Carbomer (high molecular weight, cross linked polyacrylic acid polymers). Preferably, emulsifiers or thickener or gelling agent are selected from the group consisting of cetostearyl alcohol, stearic acid, Sepineo P 600, Carbopol, Carbomer, magnesium stearate, sodium lauryl sulfate, triethanolamine, and magnesium aluminum silicate. The excipients disclosed herein above can also be used as gelling agent or as thickeners for topical compositions.

Moisturizers include, but are not limited to, glycerol, propylene glycol, and sorbitol.

Antioxidants include, but are not limited to, water soluble antioxidants, lipid-soluble antioxidants, vitamin C, vitamin C palmitate, propyl gallate, vitamin E (tocopherol), tert-butyl ether-hydroxybenzoate fennel, 2,6 di-tert-butyl-p-cresol, BHA, BHT, or mixtures of one or more antioxidants.

Lubricants include, but are not limited to, urea, magnesium stearate, sodium lauryl sulfate, polyethylene glycol, and silica gel powder.

Preservatives include, but are not limited to, chloro-m-cresol, citric acid, disodium edetate, ethoxylated alcohol, glycerin, 1,2,6-hexanetriol, methylparaben, parabens, potassium, sorbate, propyl gallate, propylene glycol, propyl paraben, sodium bisulfate, sodium citrate, butyl paraben, sodium metabisulfite, chlorocresol, sorbic acid, tannic acid, zinc stearate, butylated hydroxytoluene, butylated hydroxyanisole, benzoic acid, salicylic acid, propyl paraben, dichlorobenzyl alcohol, formaldehyde, alpha-tocopherol, sodium ascorbate, ascorbic acid, ascorbyl palmitate phenol, m-cresol, bisphenol, cetrimide, benzalkonium chloride, sorbic acid, phenoxyethanol, and benzoyl peroxide. Preferably, preservatives are selected from the group consisting of hydroxylethyl benzene, hydroxylmethyl benzene, phenoxyethanol, chlorocresol, propyl paraben, and methyl paraben. Antioxidants such as but not limited to BHA, BHT, ascorbic acids and others.

In an embodiment of the invention, topical composition of present invention has excipients that help adjusting the pH of the composition. The pH of the topical compositions may be adjusted between from about 3 to about 8 to provide a non-irritating composition. Such agents include many pharmaceutically acceptable acids, bases and buffers. Suitable acids may include one or more of hydrochloric acid, phosphoric acid and lactic acid. Suitable bases may include one or more of diethanolamine, triethanolamine and sodium hydroxide. Suitable buffers may include phosphates, such as monobasic sodium phosphate, dibasic sodium phosphate, lactates and citrates.

### EXAMPLES

The following experimental details are set forth to aid in an understanding of the invention, and are not intended, and should not be construed, to limit in any way the invention set forth in the claims that follow thereafter. A person skilled in the art will readily recognize the various modifications and variations that may be performed without altering the scope of the present invention. Such modifications and variations are encompassed within the scope of the invention and the examples do not in any way limit the scope of the invention.

### Example 1: Topical Composition of Apremilast

| Component | % w/w |
|---|---|
| Apremilast | 5 |
| DMSO | 20 |
| PEG 400 | 25 |
| Propylene glycol | 12.5 |
| Carbopol ® 941 | 1.5 |
| pH adjusting agents | QS pH 5 to 6 |
| Glycerin | QSAD 100 |

### Manufacturing Process:

a) Approximately 100% of DMSO is weighed into a stainless steel vessel; propylene glycol and polyethylene glycol 400 are added and stirred with a propeller mixer.
b) While stirring is continued, Apremilast is added to Step a) and mixed until dissolved.
c) While stirring is continued, Carbopol® 941 is added slowly to Step b) and mixed vigorously at room temperature until a uniform and lump free dispersion is achieved.
d) While stirring is continued, 80% of glycerin is added slowly to Step c) and the contents are mixed contents at room temperature until a uniform dispersion is achieved.
e) While stirring is continued, sufficient pH adjusting agent is added to the Step d) mixture to achieve a pH of 5.0 to 6.0 (±0.5).
f) The remaining glycerin is then added to step e) and mixed until uniform dispersion is achieved.
g) The Dispersion is transferred to a storage vessel and filled.

### Example 2: Topical Composition of Apremilast

| Component | % w/w |
|---|---|
| Apremilast | 0.5 |
| DMSO | 30 |
| PEG 400 | 5 |
| Propylene glycol | 13.3 |
| Carbopol ® 941 | 1.2 |
| pH adjusting agents | QS pH 5 to 6 |
| Glycerin | QSAD 100 |

### Manufacturing Process:

a) Approximately 100% of DMSO is weighed into a stainless steel vessel; propylene glycol and polyethylene glycol 400 are added and stirred with a propeller mixer.
b) While stirring is continued, Apremilast is added to Step a) & mixed until dissolved.
c) While stirring is continued, Carbopol® 941 is added slowly to Step b) & mixed vigorously at room temperature until a uniform & lump free dispersion is achieved.
d) While stirring is continued, 80% of glycerin is added slowly to Step c) & the contents are mixed at room temperature until a uniform dispersion achieved.
e) While stirring is continued, sufficient pH adjusting agent is added to the resulting mixture of Step d) to achieve a pH of 5.0 to 6.0 (±0.5).
f) The remaining glycerin is added to resultant from step e) and mixed until uniform dispersion is achieved.
g) The Dispersion is transferred to a storage vessel and filled.

### Example 3: Topical Composition of Apremilast

| Component | % w/w |
|---|---|
| Apremilast | 0.5 |
| White Soft Paraffin | 90 |
| Liquid Paraffin | 7 |
| Sorbitan sesquioleate | 0.5 |
| Menthol | 1 |
| DMSO | 1 |

### Manufacturing Process:

a) White soft paraffin and sorbitan sesquioleate are added into a melting vessel and the contents are melted at 75° C.
b) The mixture of Step (a) are transferred to Becomix and the contents are mixed at 10 rpm and cooled to 50° C.
c) Apremilast is dissolved in DMSO in a separate vessel. The solution is dispersed in liquid paraffin maintained at 60° C. using a water bath, and the contents are homogenized using a homogenizer.
d) With continuous stirring, Step c) to Step b) is mixed vigorously at room temperature until a uniform dispersion is achieved.
e) The dispersion is homogenized under vacuum at 0.4-0.6 bar at 10 rpm.
f) The Dispersion is transferred to a storage vessel and filled.

### Example 4: Topical Composition of Apremilast

| Component | % w/w |
|---|---|
| Apremilast | 0.5 |
| Oleyl Alcohol | 10 |
| White soft Paraffin | 84.7 |
| Hard Paraffin | 0.3 |
| Microcrystalline wax | 3.5 |
| DMSO | 1 |

### Manufacturing Process:

a) White soft paraffin and microcrystalline wax are added into a melting vessel and melted by heating to 70° C.
b) Mixture is transferred to Becomix, and maintained at 40° C.-45° C.
c) With continuous stirring, oleyl alcohol is added to the base obtained in Step b) and maintained at 40° C.-45° C.
d) Apremilast is dissolved in DMSO and added to the melt obtained in Step c), and homogenized for 5 minutes.
e) The product of Step (d) is allowed to cool to 30° C. and transferred to storage vessel and filled.

### Example 5: Topical Composition of Apremilast

| Component | % w/w |
|---|---|
| Apremilast | 0.5 |
| Methyl Paraben | 0.025 |
| Propyl Paraben | 0.015 |
| Sodium Lauryl Sulphate | 1 |
| DMSO | 1 |
| Propylene Glycol | 12 |
| Stearyl alcohol | 25 |
| White Petrolatum | 25 |
| Water | 36 |

### Manufacturing Process:

a) Stearyl alcohol and white petrolatum are added on a steam bath into a melting vessel and heated to about 75° C.
b) Apremilast is dissolved in DMSO; and the other ingredients in purified water; and warmed to about 75° C.
c) All ingredients are mixed together and stirred until the mixture congeals.
d) The mixture is transferred to a storage vessel and filled.

### Example 6: Topical Composition of Apremilast

| Component | % w/w |
|---|---|
| Apremilast | 0.5 |
| White wax | 12 |
| Cetyl esters wax | 12 |
| Mineral Oil | 40 |
| DMSO | 15 |
| Sodium Borate | 0.5 |
| Water | 20 |

### Manufacturing Process:

a) White wax and cetyl ester wax are added into a melting vessel and the components are melted at 70° C.
b) Mineral oil is added to the mix obtained in Step a) and further mixed until uniform mixture is obtained.
c) Apremilast is dissolved in DMSO & added to the mixture obtained in Step b).
d) Water and sodium borate is added to the mix of Step c).
e) While continuous mixing, water phase is slowly added to the oil phase.
f) The mixture is transferred to a storage vessel and filled.

### Example 7: Topical Composition of Apremilast

| Component | % w/w |
|---|---|
| Apremilast | 0.5 |
| Propylene Glycol | 48.025 |
| Glyceryl Monostearate | 5 |
| Cetostearyl alcohol | 4 |
| White wax | 0.6 |
| Chlorocresol | 0.075 |
| Water | 40 |
| DMSO | 1 |

### Manufacturing Process:

a) White wax, cetostearyl alcohol, and glyceryl monostearate are added into a melting vessel and mixed continuously while heating to 75° C. DMSO is added and mixed until uniform.
b) Apremilast is added to the mix obtained in Step a) and the temperature is maintained at 75° C.
c) Water and propylene glycol are added into a melting vessel and the contents are heated to 61-65° C.
d) Chloro cresol is added to the water phase & mixed until uniform mixture obtained.
e) Water phase is slowly added to the oil phase with continuous stirring.
f) The mixture is transferred to a storage vessel and filled.

### Example 8: Topical Composition of Apremilast

| Component | % w/w |
|---|---|
| Apremilast | 2.00 |
| Dimethyl Sulfoxide | 30.00 |
| Carbopol 974 P | 1.20 |
| Glycerin | 66.70 |
| Methyl Paraben | 0.10 |

### Manufacturing Process:

a) Approximately 100% of DMSO is weighed into a stainless steel vessel; Apremilast and methyl paraben are added to it and mixed until dissolved.
b) While stirring is continued, Carbopol 974 P is added slowly to Step a) & mixed vigorously at room temperature until a uniform and lump free dispersion is achieved.
c) While stirring is continued, 80% of glycerin is added slowly to Step b) & the contents are mixed at room temperature until a uniform dispersion achieved.
d) While stirring is continued, sufficient pH adjusting agent is added to the mixture to achieve a pH of 5.0 to 6.0 (±0.5).
e) The remaining glycerin is added & mixed until uniform dispersion achieved.
f) The Dispersion is transferred to a storage vessel and filled.

### Example 9: Topical Composition of Apremilast

| Component | % w/w |
|---|---|
| Apremilast | 2.00 |
| Dimethyl Sulfoxide | 15.00 |
| Carbopol 974 P | 1.20 |
| Glycerin | 79.70 |
| Ethanol | 2.00 |
| Methyl Paraben | 0.10 |

a) Approximately 100% of DMSO is weighed into a stainless steel vessel; ethanol and methyl paraben are added and stirred with a propeller mixer.
b) While stirring is continued, Apremilast is added to Step a) & mixed until dissolved.
c) While stirring is continued, Carbopol 974 P is added slowly to Step b) & mixed vigorously at room temperature until a uniform & lump free dispersion achieved.
d) While stirring is continued, 80% of glycerin is added slowly to Step c) & the contents are mixed at room temperature until a uniform dispersion achieved.
e) While stirring is continued, sufficient pH adjusting agent is added to the mixture to achieve a pH of 5.0 to 6.0 (±0.5).
f) The remaining glycerin is added & mixed until uniform dispersion achieved.
g) The Dispersion is transferred to a storage vessel and filled.

### Example 9.1: Topical Composition of Apremilast

| Component | % w/w |
|---|---|
| Apremilast | 2.00 |
| Dimethyl Sulfoxide | 15.70 |
| Carbopol 974 P | 0.5 |
| Glycerin | 79.70 |
| Ethanol | 2.00 |
| Methyl Paraben | 0.10 |

a) Approximately 100% of DMSO is weighed into a stainless steel vessel; ethanol and methyl paraben are added and stirred with a propeller mixer.
b) While stirring is continued, Apremilast is added to Step a) and mixed until dissolved.
c) While stirring is continued, Carbopol 974 P is added slowly to Step b) & mixed vigorously at room temperature until a uniform and lump free dispersion is achieved.
d) While stirring is continued, 80% of glycerin is added slowly to Step c) & the contents are mixed at room temperature until a uniform dispersion is achieved.
e) While stirring is continued, sufficient pH adjusting agent is added to the mixture to achieve a pH of 5.0 to 6.0 (±0.5).
f) The remaining glycerin is added & mixed until uniform dispersion achieved.
g) The Dispersion is transferred to a storage vessel and filled.

### Example 9.2: Preparation of Topical Gel

Step-1: Purified water, glycerin and propylene were mixed together;
Step-2: Carbopol 10NF (1-2.4 parts) dispersed uniformly in the step 1) mixture under continuous stirring;
Step 3: Apremilast (micronized/un-micronized) was dissolved in DMSO and is added to carbopol dispersion under stirring;
Step 4: Methyl paraben and propyl paraben were dissolved in absolute ethanol and is added to resultant mixture after step 3); and
Step 5: pH of the gel was adjusted with sodium hydroxide (NaOH) solution.

6 samples ATG1-ATG6 were prepared using the process as above, following which the physical properties and diffusion data of the formulation was evaluated for efficacy.

TABLE A
Diffusion data and physical properties of sample ATG1-ATG6:

| | AIZ115-1749- | AIZ115-1802- | AIZ115-1802- | AIZ115-1802- | AIZ115-1802- |
|---|---|---|---|---|---|
| Batch no | 082 | 108 | 110 | 112 | 107 |
| Formulation | ATG1 | ATG2 | ATG3 | ATG4 | ATG5 |
| code | | | | | |
| Description | with 2.4 | with 1 | without | without | with 1 part % |
| | parts % | part % | DMSO & | DMSO & | carbopol |
| | carbopol | carbopol | unmicronized | micronized | |
| | | | API | API | |
| Strength | 4% w/w | 4% w/w | 4% w/w | 4% w/w | 2% w/w |

Physical Properties

| Description | White to off white viscous gel | | | | |
|---|---|---|---|---|---|
| pH | 5.82 | 6.07 | 6.19 | 6.53 | 5.81 |
| Viscosity (cps) | 5147 | 1416 | 1237 | 853 | 1303 |
| Assay (%) | 102.4 | 101.3 | 100.1 | 99.9 | 100.4 |

Diffusion Data

| | | | | | |
|---|---|---|---|---|---|
| Avg. flux, | 0.965 | 1.5 | 0.67 | 0.86 | 0.73 |
| μg/sq · cm/hr | | | | | |
| Avg. | 106.6 | 173.1 | 96.0 | 114.9 | 96.8 |
| cumulative DR, | | | | | |
| μg (after 48 hrs) | | | | | |
| Avg. % DR | 0.79 | 1.4 | 0.78 | 0.95 | 1.7 |
| (after 48 hrs) | | | | | |

TABLE B
Ex vivo diffusion date of Apremilast Topical Gel 2% & 4% w/w

Batch Code

| | |
|---|---|
| ATG2 | ATG5 |

Batch no

| | |
|---|---|
| AIZ115-1802-108 | AIZ115-1802-107 |

Strength

| | |
|---|---|
| 4% w/w | 2% w/w |

Batch size

| | 7000 g | 7000 g |
|---|---|---|
| Avg. flux, μg/sq · cm/hr | 0.11 | 0.03 |
| avg. cumulative DR, μg | 19.69 | 12.29 |
| (after 48 hrs) | | |
| Avg. % DR | 0.14 | 0.18 |
| (after 48 hrs) | | |

### Example 10: Experimental Data Showing Impact on Diffusion Rate of the Composition

### Example 10.1: Comparison of Diffusion Rates of the Compositions without DMSO and with DMSO: (Unmicronized API)

Test formulations were prepared in different batches with and without DMSO to evaluate the carrier impact on the diffusion rate, however both the compositions have other critical components same in both the formulation. The test compositions were prepared with Unmicronized API and 0.5% Carbopol 10 NF:

TABLE 1

| | Batch Code | |
|---|---|---|
| | ATG6 | ATG3 |
| | Batch no | |
| | AIZ115-1749-096 | AIZ115-1802-110 |
| | Strength | |
| | 4% w/w | 4% w/w |
| | Description | |
| | Batch with 0.5% | Batch with 0.5% |
| | carbopol 10NF, with | carbopol, without |
| | DMSO and un- | DMSO and un- |
| | micronized API | micronized API |
| | Batch size | |
| | 100 g | 100 g |
| Avg. flux, μg/sq · cm/hr | 1.576 | 0.67 |
| avg. cumulative DR, μg | 181.5 | 96.00 |
| (after 48 hrs) | | |
| Avg. % DR | 1.30 | 0.78 |
| (after 48 hrs) | | |

Test results lead to an inference that the compositions without DMSO have a drastic change in Diffusion rate compared to compositions in presence of DMSO.

### Example 10.2: Comparison of Diffusion Rate of Compositions with Carbopol 0.5% and 1.2% (Unmicronized API)

Test formulations were prepared in different batches with varying concentration of Carbopol and their impact on the diffusion rate was evaluated. Remaining components were kept constant as per the formula:

TABLE 2

| | Batch Code | | |
|---|---|---|---|
| | ATG1 | ATG5 | ATG2 |
| | Batch no | | |
| | AIZ115-1749-082 | AIZ115-1802-107 | AIZ115-1802-108 |
| | Strength | | |
| | 4% w/w | 2% w/w | 4% w/w |
| | Description | | |
| | Batch with | Batch with 0.5% | Batch with |
| | 1.2% carbopol | carbopol 10NF | 0.5% carbopol |
| | 10NF | (Bio batch) | 10NF |
| | Batch size | | |
| | 270 g | 7000 g | 7000 g |
| Avg. flux, | 0.965 | 0.73 | 1.47 |
| μg/sq · cm/hr | | | |
| avg. cumulative DR, | 106.6 | 96.80 | 173.1 |
| μg (after 48 hrs) | | | |
| Avg. % DR | 0.79 | 1.7 | 1.4 |
| (after 48 hrs) | | | |

Test results lead to an inference that the compositions with increasing the carbopol concentration i.e., from 0.5-1.2% have a gradual decrease in diffusion rate.

### Example 10.3: Diffusion Data of Compositions with Micronized and Unmicronized API

Test formulations were prepared in different batches with micronized API and un-micronized API to evaluate the impact:

TABLE 3

| | Batch Code | |
|---|---|---|
| | ATG3 | ATG4 |
| | Batch no | |
| | AIZ115-1802-110 | AIZ115-1802-112 |
| | Strength | |
| | 4% w/w | 4% w/w |
| | Description | |
| | Batch with 0.5% | Batch with 0.5% |
| | carbopol, without DMSO | carbopol, without DMSO |
| | and un-micronized API | and micronized API |
| | Batch size | |
| | 100 g | 100 g |
| Avg. flux, | 0.67 | 0.86 |
| μg/sq · cm/hr | | |
| avg. cumulative DR, | 96 | 114.9 |
| μg (after 48 hrs) | | |
| Avg. % DR | 0.78 | 0.95 |
| (after 48 hrs) | | |

Test results lead to an inference that the compositions with micronized API have comparatively better results in terms of diffusion rate which were further evaluated on varying particle size ranges from 1 μm to 500 μm.

### Example 11: Stability Data of the Compositions

Test sample were evaluated for stability under stress condition of 40° C./75% RH and 30° C./65% RH as shown in Tables 4-9. At predefined time intervals the samples were withdrawn and evaluated for appearance, pH, rheological behavior, percentage drug content and related substances (known & unknown impurities).

TABLE 4
Initial and accelerated stability data of the 4% w/w Apremilast topical gel prepared using 1.2%
w/w carbopol

| Batch No. & Size | AIZ115-1612-050 (1200 g) | | | | |
|---|---|---|---|---|---|
| Strength & Pack | 4% w/w (Laminated tube) | | | | |
| Condition | Initial | 1 Month | 2 Month | 3 Month | 6 Month |
| Station | RT | 40° C./75% | 40° C./75% | 40° C./75% | 40° C./75% |
| | | RH | RH | RH | RH |

Physical parameters

| S No | Parameter | Remark | Remark | Remark | Remark | Remark |
|---|---|---|---|---|---|---|
| 1 | Description | White to off white viscous gel | | | | |
| 2 | pH | 4.84 | 4.97 | 4.86 | 4.98 | 4.60 |
| 3 | Assay (%) | 100.9 | 101.2 | 102.0 | 100.2 | 99.8 |
| 4 | Viscosity (cps) | 4078 | 3891 | 3765 | 3689 | 3412 |

Related substances (% w/w)

| | | | | | | |
|---|---|---|---|---|---|---|
| 1 | Open diamide/ | 0.01 | 0.01 | 0.02 | 0.12 | 0.16 |
| | Impurity-H | | | | | |
| 2 | Desacetyl/ | 0.04 | 0.08 | 0.10 | 0.12 | 0.19 |
| | Impurity-B | | | | | |
| 3 | Single max. | 0.06 (1.61) | 0.05 (1.62) | 0.06 (1.61) | 0.06 (1.65) | 0.06 (1.65) |
| | Unknown(RRT) | | | | | |
| 4 | Total Impurities | 0.13 | 0.18 | 0.26 | 0.36 | 0.50 |

TABLE 5
Initial and Real time stability data of the 2% w/w Apremilast topical gel

| | | Batch No. & size | |
|---|---|---|---|
| | | 500 g (AIZ115-1612-073) | |
| | | Strength & Pack | |
| | | 2% w/w (Laminated tube) | |
| | | Condition | |
| | | | 3 Month/30° C./ |
| | | Initial/RT | 65% RH |
| S No | Parameter | Remark | Remark |

Physical parameters

| | | | |
|---|---|---|---|
| 1 | Description | White to off white | White to off white |
| | | viscous gel | viscous gel |
| 2 | pH | 5.64 | 5.62 |
| 3 | Assay (%) | 99.4 | 101.1 |
| 4 | Viscosity (cps) | 6600 | 6300 |

Related substances (% w/w)

| | | | |
|---|---|---|---|
| 1 | Open diamide/Impurity-H | 0.03 | 0.08 |
| 2 | Desacetyl/Impurity-B | 0.03 | 0.02 |
| 3 | Single maximum Unknown(RRT) | 0.09 (1.58) | 0.04(1.64) |
| 4 | Total Impurities | 0.23 | 0.14 |

TABLE 6
Initial and Real time stability data of the 4% w/w Apremilast topical gel

| | | Batch No. & size | | |
|---|---|---|---|---|
| | | 1200 g (AIZ115-1612-053) | | |
| | | Strength & Pack | | |
| | | 4% w/w (Laminated tube) | | |
| | | Condition | | |
| | | Initial/RT | 3 Month | 6 Month/ |
| | | Station | | |
| | | RT | 30° C./65% RH | 30° C./65% RH |
| S No | Parameter | Remark | Remark | Remark |

Physical parameters

| 1 | Description | White to off white viscous gel | | |
|---|---|---|---|---|
| 2 | pH | 5.38 | 5.08 | 4.81 |
| 3 | Assay (%) | 104.8 | 105.7 | 100.3 |
| 4 | Viscosity (cps) | 7234 | 6500 | 6390 |

Related substances (% w/w)

| | | | | |
|---|---|---|---|---|
| 1 | Open diamide/Impurity-H | 0.02 | 0.03 | 0.05 |
| 2 | Desacetyl/Impurity-B | 0.04 | 0.04 | 0.06 |
| 3 | Single maximum Unknown(RRT) | 0.06 (1.62) | 0.05 (1.65) | 0.06 (1.65) |
| 4 | Total Impurities | 0.12 | 0.12 | 0.17 |

TABLE 7
Initial and accelerated stability data of the 2% w/w Apremilast topical gel prepared using 1.2%
w/w carbopol

| Batch No. & Size | AIZ115-1612-073 (500 g) | | | | |
|---|---|---|---|---|---|
| Strength & Pack | 2% w/w (Laminated tube) | | | | |
| Condition | Initial/RT | 1 Month/ | 2 Month/ | 3 Month/ | 6 Month/ |
| | | 40° C./75% | 40° C./75% | 40° C./75% | 40° C./75% |
| | | RH | RH | RH | RH |

Physical parameters

| S No | Parameter | Remark | Remark | Remark | Remark | Remark |
|---|---|---|---|---|---|---|
| 1 | Description | White to off white viscous gel | | | | |
| 2 | pH | 5.64 | 5.6 | 5.6 | 5.59 | 5.62 |
| 3 | Assay (%) | 99.4 | 100.3 | 99.2 | 99.8 | 98.6 |
| 4 | Viscosity (cps) | 6600 | 6600 | 6600 | 6600 | 5684 |

Related substances (% w/w)

| | | | | | | |
|---|---|---|---|---|---|---|
| 1 | Open diamide/ | 0.03 | 0.12 | 0.16 | 0.22 | 0.16 |
| | Impurity-H | | | | | |
| 2 | Desacetyl/ | 0.03 | 0.06 | 0.09 | 0.12 | 0.1 |
| | Impurity-B | | | | | |
| 3 | Single max. | 0.09(1.58) | 0.06(0.14) | 0.11(0.14) | 0.15(0.14) | 0.27(0.14) |
| | Unknown(RRT) | | | | | |
| 4 | Total Impurities | 0.28 | 0.34 | 0.50 | 0.71 | 0.9 |

TABLE 8
Initial and accelerated stability data of the 4% w/w Apremilast topical gel prepared using 0.5%
w/w carbopol

| Batch No. & Size | AIZ115-1802-108 (7000 g) | | | | |
|---|---|---|---|---|---|
| Strength & Pack | 4% w/w (Laminated tube) | | | | |
| Condition | RT | 40/75% RH | | | |
| Station | Initial | 1 Month | 2 Month | 3 Month | 6 Month |

Physical parameters

| S No | Parameter | Remark | Remark | Remark | Remark | Remark |
|---|---|---|---|---|---|---|
| 1 | Description | White to off white viscous gel | | | | |
| 2 | pH | 6.06 | 6.17 | 6.15 | 5.71 | 5.65 |
| 3 | Assay (%) | 101.3 | 102.0 | 100.8 | 101.9 | 99.4 |
| 4 | Viscosity (cps) | 1416 | 1181 | 1116 | 1050 | 375 |

Related substances (% w/w)

| | | | | | | |
|---|---|---|---|---|---|---|
| 1 | Impurity-H | 0.21 | 0.28 | 0.35 | 0.27 | 0.22 |
| 2 | Impurity-B | 0.04 | 0.05 | 0.09 | 0.2 | 0.21 |
| 3 | Single max. | 0.08(1.63) | 0.16(0.28) | 0.18(0.28) | 0.16(0.14) | 0.29(0.54) |
| | Unknown(RRT) | | | | | |
| 4 | Total Impurities | 0.4 | 0.63 | 0.85 | 1.08 | 1.29 |

TABLE 9
Initial and accelerated stability data of the 2% w/w Apremilast topical gel prepared using 0.5%
w/w carbopol

| Batch No. & Size | AIZ115-1802-107 (7000 g) | | | | |
|---|---|---|---|---|---|
| Strength & Pack | 2% w/w (Lami tubes without seal) | | | | |
| Condition | RT | 40/75% RH | | | |
| Station | Initial | 1 Month | 2 Month | 3 Month | 6 Month |

Physical parameters

| S No | Parameter | Remark | Remark | Remark | Remark | Remark |
|---|---|---|---|---|---|---|
| 1 | Description | White to off white viscous gel | | | | |
| 2 | pH | 5.82 | 5.85 | 5.82 | 5.41 | 5.39 |
| 3 | Assay (%) | 100.4 | 100.3 | 98.4 | 96.8 | 95.4 |
| 4 | Viscosity (cps) | 1303 | 1144 | 1050 | 881 | 319 |

Related substances (% w/w)

| | | | | | | |
|---|---|---|---|---|---|---|
| 1 | Impurity-H | 0.34 | 0.35 | 0.41 | 0.32 | 0.31 |
| 2 | Impurity-B | 0.04 | 0.06 | 0.17 | 0.28 | 0.4 |
| 3 | Single max. | 0.08(0.28) | 0.2(0.28) | 0.24(0.28) | 0.23(0.14) | 0.48(0.54) |
| | Unknown(RRT) | | | | | |
| 4 | Total Impurities | 0.56 | 0.85 | 1.09 | 1.43 | 2.15 |

The total impurity level in the above analysed samples of Table 4-9 were found to be within the limits, thus the instant composition is stable and suitable for storage and further application.

### Example 12: Study to Evaluate the Effect of Various Factors Like Carbopol Concentration, Permeation Enhancer Concentration, Gel Strength and Apremilast Micronization on Apremilast Topical Gel Composition

A. Preparation of Topical Gel: Gels were prepared as shown in Examples and more specifically in Example 9.3.
B. In Vitro drug release study: In vitro drug release study was performed in vertical Franz diffusion cell (Orchid Scientific, EM FDC06) using ethanolic phosphate buffer [Phosphate buffer pH 7.4: Ethanol (70:30)] as receptor fluid & “Strat M” as “semi permeable membrane”. The test formulation was applied on Strat M surface & experiment was carried out under non-occlusive condition. The receptor compartment (20 ml) was filled with receptor medium which was stirred continuously with a magnetic stirrer at 600 rpm & temperature 37±0.2° C. At each predetermined time intervals (2, 4, 6, 8, 9, 12, 24 and 48 h), aliquot receptor fluid was withdrawn & same volume was replaced with fresh medium. The samples were suitably diluted and analyzed using HPLC technique.
C. Ex vivo drug release study: Ex vivo drug release study was performed for ATG2 and ATG5 formulations using pork ear skin as semi permeable membrane. Results are shown in Table B.

### C.1: Preparation of Skin for Ex Vivo Skin Permeation Study:

Porcine ear skin was obtained from the slaughter house after sacrificing the animal within 1 h. The hair was removed from upper portion of skin surface using an animal hair clipper & the fatty layer adhered to the dermis side was removed by surgical scalpel. Then the skin was rinsed with deionized water & packed in an aluminum foil followed by storing the skin samples in freezer (−20° C.) & using next day.

### C.2: Ex Vivo Permeation Study in Franz Diffusion Cell:

Ex vivo permeation study was carried out in vertical Franz diffusion cell.
The donor compartment was open at the top and was exposed to atmosphere (Non-occlusive nature).
The excised porcine ear skin was mounted between the donor and receptor compartments of the diffusion cell in such a way that the stratum corneum faced towards donor compartment.
Magnetic stirrer bars were added to the receptor chambers and filled with the receptor medium.
The entire setup was placed over magnetic stirrer and the temperature was maintained at 37±0.5° C.
Before mounting the skin in diffusion cell, the skin was kept for hydration in receptor fluid for 1 hr.

The specific quantity of the gel to be analyzed was placed in donor compartment & diffusion was carried out for 48 hrs. At predefined time intervals (2, 4, 6, 8, 9, 12, 24 & 48 h), aliquot samples were withdrawn from receptor fluid & fresh samples were replaced as described earlier. The amount of drug permeated was measured using HPLC.

### C.3: Analysis of Ex Vivo Diffusion Data:

The cumulative amount of Apremilast permeated per unit area of pork ear skin (μg/cm²) was calculated and plotted against time. The steady state flux (Jss, μg/cm²/min) was calculated from the slope of the linear portion of the plot and the permeability coefficient (P) expressed as cm²/hr were calculated according to the following equations.

$$Jss = \frac{dQ}{dt} / A$$

$$P = \frac{dQ}{dt} \times \frac{1}{ACd}$$

Where,
A is the diffusion area of the patch (2.95 cm²),
dQ/dt is the slope of the linear region in the cumulative amount of Apremilast permeated per unit area-time plot and lag time was determined by extrapolating the linear portion of the cumulative amount permeated per unit surface area-time plot to the abscissa (x-intercept).
D. Evaluation of topical gel composition of Apremilast: The physical properties & diffusion data of topical formulation ATG1-ATG6 were evaluated as shown in above Table A & B, and the data were satisfactory and the composition was confirmed to be suitable for application. Further the evaluation summary is provided as below:

### D.1: Physical Evaluation:

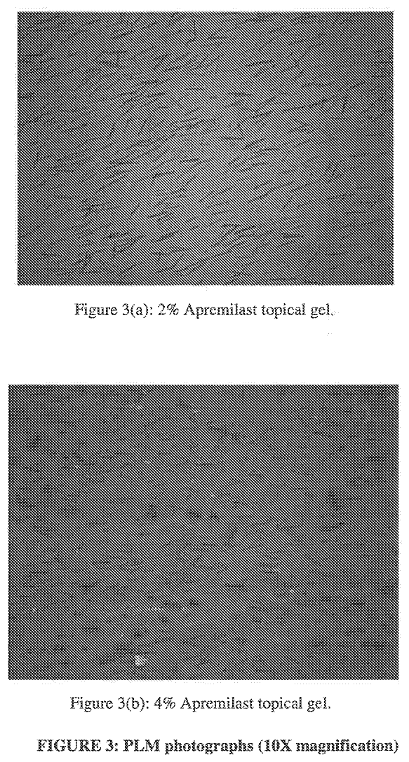

All the developed hydrophilic gels were white to off white viscous gels with a smooth homogenous texture and glossy appearance. The dispersed crystals appeared as small rod shaper crystals as shown in FIGS. 3(a) and 3(b).

### D.2: pH:

The pH of all formulation was found between pH 6.0±0.5 and the results were reported in Table A. The pH values seemed suitable for skin application as the results are more or less equal to the skin pH and this may confirms the less risk of irritation upon application to the skin.

### D.3: Rheological Determination:

The viscosity of the prepared gels was measured using cone and plate viscometer and reported in Table A. During the viscosity measurement in all the formulations, it was observed that viscosity decreased on increasing the shear. Viscosity of the ATG1 formulation was more than the ATG2 formulation. This was attributed to be the effect of carbopol concentration. It was observed that the Carbopol concentration in the gel is directly proportional to the viscosity of the gel.

### D.4: Determination of Drug Content:

The drug content in formulated gel was measured and reported in Table A. The drug content values were found within the acceptance range (Acceptance range: 100±5% w/w) and this also confirms the uniform distribution of the drug in the gels.

### D.5: Effect of Carbopol Concentration on Drug Release:

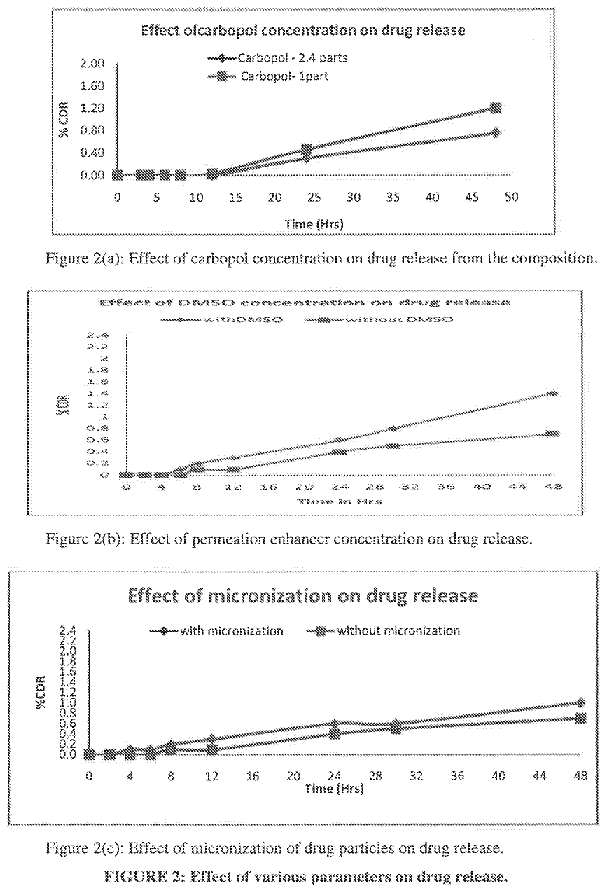

The drug release from ATG1 and ATG2 was studied and more drug permeation was observed from ATG2 than ATG1 across the “Strat M” which could be attributed to the high concentration of carbopol used in ATG1. Diffusion profiles of both formulations presented in FIG. 2(a).

### D.6: Effect of Permeation Enhancer Concentration on Drug Release:

The cumulative percent drug release across the “Strat M” from ATG2 and ATG3 formulations was compared to study the effect of DMSO on drug release. It was observed that the drug release from ATG3 is approximately half of the drug released from ATG2. This indicates DMSO is playing a significant role in drug penetration through membrane. Diffusion profiles of both formulations presented in FIG. 2(b).

### D.7: Effect of Micronization on Drug Release:

Drug release from ATG3 and ATG4 was compared across the “Strat M” to study the effect of micronization of API on drug release and the results reported that there is no influence of micronization of API on drug release. This could be attributed due to solubilization of Apremilast in DMSO and adding to the water phase during the manufacturing of the gels. However the drug is precipitated as small less dense crystals in both the formulations while adding drug solution to the water phase and was confirmed by the Polarized light microscope (as shown in FIGS. 2(c), 3(a) and 3(b)).

### D.8: Effect of Strength on Drug Release:

The drug release profiles of 2% and 4% w/w Apremilast gels (ATG2 & ATG5) across the “Strat M” was compared with each other to study the effect of strength of the gel on drug release. The release profiles showed that decrease in drug release with increased strength of the gel. It was concluded that the drug to the penetration enhancer ratio might be contributing to the release; higher the release with high ratio of drug to penetration enhancer (1:5) and lower release with lower ratio (1:2.5). Thus this effect can be neutralized with increasing the concentration of penetration enhancer in the higher strength dosage forms for 0.5-10% Apremilast gels.

Accordingly based on the evaluation study done on various factors like effect of carbopol concentration, DMSO presence, micronization of API and strength of gel, along with other physiochemical parameters various formulation as shown in few of the contemplated Examples 1-9 were made and evaluated. The applicant concluded that the careful selection of concentration of carbopol and strategic control of the viscosity of the gel plays crucial role in providing an improved diffusion of the drug from the topical composition on to the target area of application in presence of permeation enhancer. Although the proposed compositions of present invention are found to be more stable and have characteristically enhanced diffusion of drug from the topical composition. However the choice of components and process of preparation plays a critical role in the overall stability & storage stability of the composition with controlled amount of total and any known and unknown impurities which are under study.

We claim:

1. An improved topical pharmaceutical composition of Apremilast, comprising Apremilast or its pharmaceutically acceptable salt with at least one high molecular weight, cross linked polyacrylic acid polymer, and pharmaceutically acceptable excipients, for alleviating the symptoms of psoriasis or psoriatic arthritis.

2. The topical pharmaceutical composition as claimed in claim 1, wherein the high molecular weight, cross linked polyacrylic acid polymer is a gelling agent or a thickener or an emulsifier.

3. The topical pharmaceutical composition as claimed in claim 2, wherein the gelling agent or a thickener or an emulsifier is selected from the group comprising of oleyl alcohol, polyoxyethylene oleyl ether, PEG-40 stearate, ceteareth-12, ceteareth-20, ceteareth-30, glyceryl stearate, PEG-100 stearate, methyl myristate, isopropyl myristate, synthetic and semi-synthetic commercially available gums, Arlacel 165, glyceryl stearate, polyethylene glycol (PEG)-100 stearate, steareth-2 and steareth-20, dimethicone copolyol, Polysorbate 20 (Tween 20), cetyl esters wax, Polysorbate 40 (Tween 40), Polysorbate 60 (Tween 60), Polysorbate 80 (Tween 80), lauramide, cocamide, and cocamide monoethanolamine, Phospholipid PTC, alginate, carrageenan, Glucate DO, methylcellulose, polyvinyl alcohol, Sepineo P 600, Carbopol and Carbomer or mixture thereof.

4. The topical pharmaceutical composition as claimed in claim 3, wherein the gelling agent or a thickener or an emulsifier is selected from the group comprising of Carbopol, Carbomer, cetostearyl alcohol, stearic acid, Sepineo P 600, Carbopol, Carbomer, magnesium stearate, sodium lauryl sulfate, triethanolamine, and magnesium aluminum silicate or mixture thereof.

5. The topical pharmaceutical composition as claimed in claim 1, wherein the high molecular weight, cross linked polyacrylic acid polymer is a gelling agent selected from Carbopol and Carbomer.

6. The topical pharmaceutical composition as claimed in claim 1, wherein the pharmaceutically acceptable excipient is selected from the group comprising of carrier, pharmaceutical surfactant, solubilizer or solubility enhancers, skin penetration/permeation enhancers, oil phase components, aqueous phase components, emulsifiers, moisturizers, antioxidants, vitamins, lubricants, preservatives, stabilizers and other ingredients.

7. The topical pharmaceutical composition as claimed in claim 6, wherein the skin penetration/permeation enhancers is selected from the group comprising of sulfoxides (DMSO), azones (laurocapram), pyrrolidones (2-pyrrolidone), alcohols and alkanols, oleic acids, glycols, dimethylformamide, dimethylacetamide, fatty alcohols, fatty acid esters, fatty acids, fatty alcohol ethers, terpenes, proteins or yellow fat and mixture thereof

8. The topical pharmaceutical composition as claimed in claim 1, wherein the ratio of apremilast to high molecular weight, cross linked polyacrylic acid polymer in the composition is 0.5:0.2 to 15:3, preferably 0.5:0.5 to 10:1.5.

9. The topical pharmaceutical composition as claimed in claim 1, wherein the concentration of apremilast in the composition is in the range of 0.5-15% w/w, and the Apremilast particles can be micronized or un-micronized and the concentration of at least one high molecular weight, cross linked polyacrylic acid polymer is in the range of 0.2-3.0% w/w.

10. The topical pharmaceutical composition as claimed in claim 1, wherein the composition is in the form a topical gel, cream, ointment, spray, lotion or solution.
